(19) United States
(12) Patent Application Publication
(10) Pub. No.: US 2012/0195877 A1
(43) Pub. Date: Aug. 2, 2012

(54) DEGRADABLE COMPOSITE MATERIAL CONTAINING CHITIN OR CHITOSAN

(75) Inventors: Takashi Oku, Hyogo (JP); Takeshi Noguchi, Oita (JP)

(73) Assignees: TOTAI CO., LTD., Tokyo (JP); THERMOSTABLE ENZYME LABORATORY CO., LTD., Hyogo (JP)

(21) Appl. No.: 13/501,069

(22) PCT Filed: Oct. 7, 2009
(86) PCT No.: PCT/JP2009/067522

§ 371 Date: Apr. 9, 2012

(51) Int. Cl. A61K 47/42; C12N 11/10; A61P 17/02

(52) U.S. Cl. 424/94.61; 435/178

(57) ABSTRACT

The object of the present invention is to provide a degradable composite material that contains at least one type of polysaccharide selected from chitin and chitosan, and has self-degrading capability. By supporting an enzyme capable of hydrolyzing chitin and chitosan on a molded article containing at least one type of polysaccharide selected therefrom, the polysaccharide can be slowly degraded in an environment where moisture is present such as a living body.

### TECHNICAL FIELD

The present invention relates to a degradable composite material containing at least one type of polysaccharide selected from chitin and chitosan. More specifically, the present invention relates to a degradable composite material that contains at least one type of polysaccharide selected from chitin and chitosan and that slowly degrades in an environment where moisture is present, such as a living body.

### BACKGROUND ART

Chitin and chitosan are used in a variety of medical materials because they do not adversely affect ecological systems (see Patent Document 1). For example, sheets manufactured having chitin and chitosan as a raw material are used as a wound dressing material, and fibers manufactured having chitin and chitosan as a raw material are used as a medical prosthetic material. It is known that when chitin and chitosan degrade, they become soluble saccharides that are absorbed in vivo, and it has been learned that the degradation products thereof are highly biocompatible. There are problems, however, because chitin and chitosan cannot be broken down by enzymes in the human body, so medical materials manufactured with these substances as a raw material are difficult to use in a mode that remains in the body. Medical materials to be used such that they remain in body tissues for long periods of time have the advantage of safety and ease of use because removal of the medical material that has served its purpose becomes unnecessary if it is absorbed in the body and disappears at a stage wherein damage to the tissue has healed to a certain extent after surgery.

Therefore, medical materials with even greater utility can be provided, if a self-degrading capability can be imparted to chitin and chitosan. Until now, however, no technology capable of providing chitin and chitosan with this self-degrading capability has been established.

### SUMMARY OF THE INVENTION

An object of the present invention is to provide a degradable composite material that contains at least one type of polysaccharide selected from chitin and chitosan, and has self-degrading capability.

The following invention solves the above problems by supporting an enzyme capable of hydrolyzing chitin and chitosan on a molded article containing at least one type of polysaccharide selected therefrom, the polysaccharide can be slowly degraded in an environment where moisture is present such as a living body, and that a degradable composite material having self-degrading capability can be provided thereby. In particular, by using a thermostable enzyme as the above degrading enzyme, the degrading activity thereof on the polysaccharide can be stably maintained over a long period of time, and both sustained and safe degradation of the composite material becomes possible in an environment where moisture is present. The present invention was completed by further modifications and improvements based on this knowledge.

More specifically, the present invention provides a degradable composite material with the following features:

A degradable composite material comprising a degrading enzyme that is capable of hydrolyzing at least one type of polysaccharide selected from the group consisting of chitin and chitosan and is supported on a molded article containing the polysaccharide.

The molded article containing the polysaccharide may be in the form of a sheet.

The molded article containing the above polysaccharide may be in the form of a fiber.

In one embodiment, the degrading enzyme is a thermostable chitinase that exhibits activity at 4° C. or higher and has an optimal temperature of 95° C. or higher.

In another embodiment, the degrading enzyme is a thermostable chitinase originating in Pyrococcus furiosus.

In one instance, the degradable composite material is a medical material.

The degradable composite material of any of the embodiments herein, for the manufacture of a medical material.

The medical material may be a wound dressing or medical prosthetic material.

A method for treating a wound, wherein a wound site of a patient is covered with a degradable composite material according to the present invention.

A method for suturing an incision site or a laceration site, wherein an incision site or a laceration site in a patient is sutured with the degradable composite material according to the present invention.

### ADVANTAGEOUS EFFECTS OF INVENTION

In an environment where moisture is present the degradable composite material of chitin and/or chitosan of the present invention can self-degrade into highly biocompatible soluble saccharides. As a result, the burden on the patient can be reduced because when the degradable composite material of the present invention is applied to injured tissue as a medical material, it disappears after remaining on the injured tissue for a desired period of time, and therefore removal of the medical material after surgery is unnecessary.

Moreover, by using a degrading enzyme that exhibits activity over a wide temperature range of 4° C. to 100° C. as the degrading enzyme that can hydrolyze the chitin and/or chitosan in the degradable composite material of the present invention, self-degradation can be induced in an environment where moisture is present under a variety of temperature conditions and thus this enables the present invention to be used in a broad range of applications.

In addition, by using a thermostable enzyme as the degrading enzyme in the degradable composite material of the present invention, the self-degradation capability of the degradable composite material can be stably maintained because of the high level of stability of the enzyme, and therefore sustained and stable decomposition of the degradable composite material becomes possible in an environment where moisture is present. In addition, this feature provides long-term storage stability.

### BRIEF DESCRIPTION OF THE DRAWINGS

FIG. 1 is a photograph showing the result when disintegration was evaluated in a sheet comprising chitin at a temperature of 60° C. in acetate buffer solution.

### DESCRIPTION OF EMBODIMENTS

The degradable composite material of the present invention is characterized in that a degrading enzyme that can hydrolyze at least one type of polysaccharide selected from the group consisting of chitin and chitosan is supported on a molded article containing the polysaccharide (hereinafter, sometimes simply expressed as “chitinous molded article”). The best mode for carrying out the invention is described in detail below.

In general, chitin is a polysaccharide wherein N-acetylglucosamine structural units are joined by β-1,4 glycoside linkages, but in the chitin used in the present invention less than 50% of the acetyl groups can be deacetylated. In addition, chitosan is a deacetylated polysaccharide, and the chitosan used in the present invention can include parts that have not been deacetylated provided 50% or more of the acetyl groups of chitin have been deacetylated.

The chitinous molded article used in the degradable composite material of the present invention can comprise either chitin or chitosan as the polysaccharide, or it can comprise a combination thereof.

In addition, the chitinous molded article used in the degradable composite material of the present invention can be formed of only the above polysaccharides or, as needed, can also comprise other degradable resins, additives, and the like.

The content ratio of the above polysaccharide is not particularly limited in the chitinous molded article using the degradable composite material of the present invention, but from the perspective of increasing the self-degrading capability of the degradable composite material the total weight of polysaccharide per total weight of chitinous molded article is preferably 30 wt % to 100 wt %, more preferably 50 wt % to 100 wt %, and even more preferably 70 wt % to 100 wt %.

The configuration of the chitinous molded article of the degradable composite material of the present invention can be suitably established according to the intended mode of the degradable composite material and is not particularly limited herein. Examples include a sheet-like, fiber (thread)-like, nonwoven fabric-like, cotton-like, sponge-like, etc. Among these sheet-like and fiber-like forms are preferred because they are configurations that are easy to use as medical materials, etc.

The chitinous molded article can be produced by known manufacturing methods or can be obtained as a commercial product.

The degrading enzyme used in the degradable composite material of the present invention is not particularly limited herein provided it can hydrolyze the polysaccharide and is pharmacologically acceptable. Specific examples of such degrading enzymes include chitinases, chitosanases, and the like. For example, if the polysaccharide is chitin, a chitinase is used, or if the polysaccharide is chitosan, a chitosanase is used. The degrading enzyme used in the degradable composite material of the present invention can be one type of enzyme used alone, or can be two or more enzymes used in combination.

The effective temperature of the degrading enzyme used in the present invention is not particularly limited herein provided it exhibits activity in the temperature and environment wherein the degradable composite material is to be used. Preferably, in one embodiment, the degrading enzyme exhibits activity over a broad temperature range of 4° C. to 100° C. By using an enzyme that exhibits activity in such a wide temperature range it is possible to induce self-degradation of the degradable composite material in an environment where moisture is present under a variety of temperature conditions, and the degradable composite material invention can thereby be utilized over a wide range of applications.

Furthermore, a preferred example of the degrading enzyme used in the present invention is a thermostable enzyme. The use of such a thermostable enzyme not only can impart self-degrading capability to the degradable composite material of the present invention under high temperature conditions, but also can increase storage stability thereof. An example of such a thermostable enzyme is one with an optimal temperature of 60° C. or higher, and more preferably about 90° C. A preferred example of the thermostable enzyme used in the present invention is a thermostable chitinase that exhibits activity at 4° C. or higher and has an optimal temperature of 95° C. or higher. A concrete example of such a thermostable chitinase is one belonging to Pyrococcus furiosus. A thermostable chitinase originating in Pyrococcus furiosus is already publicly known, and can be produced by the method disclosed in Japanese Patent Application Laid-open No. 2004-344160.

A preferred mode of the degradable composite material of the present invention is a chitinase supported on a molded article containing chitin; a more preferred mode is a chitinase supported on sheet-like or fiber-like molded article containing chitin; and a particularly preferred mode is a thermostable chitinase supported on a sheet-like or fiber-like molded article containing chitin.

The degrading enzyme in the degradable composite material of the present invention can be supported on the molded article containing a polysaccharide by physical adsorption or by chemical bonding. The degradable composite material of the present invention can be manufactured, for example, by applying a solution containing the degrading enzyme to the molded article containing the polysaccharide or impregnating the molded article with that solution. In addition, it can be manufactured by kneading the degrading enzyme into the molded article containing the polysaccharide.

The amount of degrading enzyme that is supported on the molded article containing the polysaccharide in the degradable composite material of the present invention is not particularly limited herein, and can be suitably established in accordance with the type of polysaccharide, mode of degradable composite material, use thereof, and the like. An example of the supported amount of degrading enzyme is about 100 U to about 100,000 U of degrading enzyme per 1 g of polysaccharide, preferably about 1,000 U to about 10,000 U, and more preferably about 2,000 U to about 5,000 U. For the unit of activity of the degrading enzyme used herein, 1 U is defined as the amount of enzymatic activity needed to release 1 μmol of N-acetylglucosamine or glucosamine in 1 minute under the conditions noted below.

Substrate solution: Chitin powder or chitosan powder is added to 200 mM acetate buffer (pH 5.6) to make 0.5 wt % and then suspended.
Degrading enzyme solution: The degrading enzyme to be measured is dissolved to a concentration of 0.8 mg/mL in distilled water.

### Measurement Protocol:

(1) First 1 mL of substrate solution is placed in a test tube and incubated for 5 min to 10 min at 37° C.
(2) Next 0.2 mL of degrading enzyme solution is added to the test tube, and the degrading enzyme is incubated under gentle agitation for 1 hour at 37° C.
(3) After the mixture has cooled, 1000 μL of DMAB (para-dimethylamino benzaldehyde) reagent is added and after heating for 20 min at 37° C., the optical absorption is measured at 585 nm to determine the increase in reducing ends and the released amount of N-acetylglucosamine or glucosamine is quantitated thereby. The DMAB reagent is prepared by adding 10 g of DMAB to 100 mL of acetic acid containing 12.5 wt % of 10 N hydrochloric acid, and diluting 10-fold with acetic acid immediately before use.

The uses of the degradable composite material of the present invention are not particularly limited herein and the present invention can be used in a variety of fields. A preferred example of the use of the degradable composite material of the present invention is a wound dressing material, medical prosthesis material, or other medical material. When the degradable composite material of the present invention is applied to the affected area, the degrading enzyme supported thereon is activated by coming into contact with the moisture present at the affected site, and self-degradation begins. Thus, because the degradable composite material of the present invention self-degrades and is absorbed in vivo, it is useful as a medical material (such as a wound dressing or medical prosthesis material) that will remain in a body tissue for a long period of time. In this case, when the degradable composite material of the present invention is used as a medical material, it can be applied to medical uses in humans or to medical uses in mammalian animals (nonhuman).

If the degradable composite material of the present invention is to be used in the form of a sheet, for example, it can be quite suitably used as a wound dressing material. If the degradable composite material of the present invention is to be used as a wound dressing material, the wounded area only needs to be covered with the wound dressing material. A wound dressing material that is applied to a wounded area is advantageous because it will degrade and be absorbed in vivo at a stage wherein the wound has healed to a certain extent, and removal after the wound has healed is unnecessary. In addition, if the degradable composite material of the present invention is used as a wound dressing material, the therapeutic effect on the wound can be increased by the gradual degradation thereof during the process of wound healing.

Moreover, if the degradable composite material of the present invention is to be used in the form of a fiber, for example, it can be quite suitably used as a medical prosthetic material. If the degradable composite material of the present invention is to be used as a medical prosthetic material, an incised or lacerated area of the patient only needs to be sutured with the medical prosthetic material. The burden on the patient can be reduced because the medical prosthetic material used for suturing an incised or lacerated area is absorbed in the body as the sutured area heals, and removal of the stitches after surgery is unnecessary.

### EXAMPLES

The present invention is described in detail below through examples, but is by no means limited thereto.

### Example 1

### 1. Preparation of Chitinase

Agitation culturing of E. coli carrying a plasmid incorporating DNA that codes for a chitinase originating in Pyrococcus furiosus (the amino acid sequence represented by SEQ ID NO: 2 of Japanese Patent Application Laid-open No. 2004-344160) was performed at 37° C. using LB culture medium (containing 10 g/L polypeptone, 5 g/L yeast extract, and 10 g/L sodium chloride), and isopropyl β-thiogalactopyranoside (IPTG) was added during the logarithmic growth phase (optical density of 0.2 to 0.4 at 600 nm) to make a final concentration of 0.2 mM. Culturing was continued unchanged overnight, and then the E. coli cells were collected by centrifugal separation (7 min at 6,000×g).

The E. coli cells recovered from 1 liter of culture liquid were suspended in 20 mL of buffer A (25 mM Tris-HCl [pH 7.5], 1 mM EDTA, 25 mM NaCl) and lysed by sonication. After lysing, a liquid extract was obtained by high-speed centrifugation (15 min at 13,000×g). The liquid extract was heated at 85° C. for 30 min, high-speed centrifugation (15 min at 13,000×g) was performed once more and the supernatant was collected.

The collected supernatant was added to a HiTrapQ anion exchange column (Amersham, 5 mL). AKTA prime (Amersham) was used for addition of the supernatant to the column and for elution. Elution was performed over a concentration gradient (25 mM to 1 M) of the salt (NaCl) contained in buffer A. The fractions wherein the target chitinase eluted were collected and the chitinase was precipitated overnight at 4° C. in 80% saturated ammonium sulfate. The precipitate was recovered by high-speed centrifugation (15 min at 13,000×g) and redissolved in buffer A. The dissolved sample was added to a HiLoad 26/60 Superdex-200pg gel filtration column (Amersham) that had been equilibrated with buffer A to remove the low molecular weight proteins and residual ammonium sulfate. The chitinase elution fractions were collected and concentrated with CentriPrep YM-10 (Amicon).

The chitinase obtained in this manner had chitinase activity of 18,000 U/g (dry weight equivalent) at 37° C.

### 2. Evaluation of Degrading Capability of Chitinase on a Sheet Comprising Chitin

A sheet comprising chitin (Beschitin W, Unitika) was cut into 20 mm×20 mm squares (approximately 0.0125 g), and the squares were immersed in 20 mL of either 50 mM acetate buffer (pH 4.5) or 50 mM Tris buffer (pH 7.4). Then 0.0006 g of chitinase obtained in the above manner was added to the two solutions, and the solutions were let stand while holding the temperature at either 37° C. or 60° C. After a fixed period of time, the sheets were removed and the amount of disintegration was checked. For comparison purposes, the same test was performed without adding chitinase.

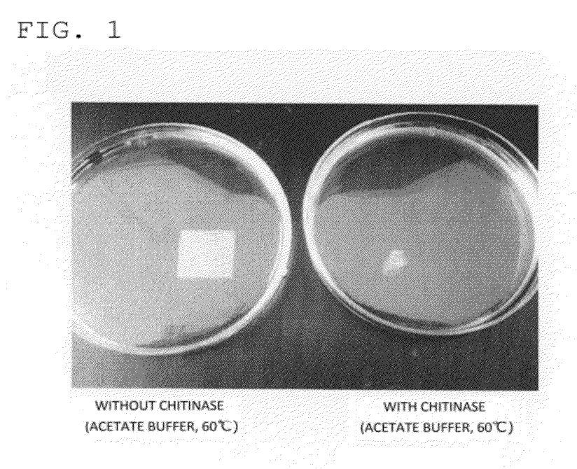

FIG. 1 shows the results of the evaluation of sheet disintegration in acetate buffer at a temperature of 60° C. As shown in FIG. 1, it is clear that the sheet comprising chitin is degraded by the co-presence of chitinase. Similar results were obtained for acetate buffer at 37° C., Tris buffer at 37° C., and Tris buffer at 60° C. The sheet comprising chitin was degraded in the presence of chitinase at 37° C., and therefore the above results confirm that a chitinous sheet whereon chitinase is supported will self-degrade in vivo and is useful in medical applications.

### 3. Manufacture of Degradable Composite Material

A sheet comprising chitin (Beschitin W, Unitika, 100 mm×120 mm, 0.4 g) was immersed at 4° C. for 30 min in 10 mL of 50 mM Tris buffer (pH 7.5) containing the chitinase obtained in the above manner at a concentration of 0.011 g/mL. Then, a chitin sheet whereon chitinase is supported (degradable composite material) was obtained by removing the sheet and drying it at 50° C.

1-10. (canceled)

11. A degradable composite material comprising:
a thermostable chitinase that exhibits activity at 4° C. or higher and has an optimal temperature of 95° C. or higher and is supported on a molded article containing at least one type of polysaccharide selected from chitin and chitosan, wherein the molded article is a medical material that is in the form of a sheet or a fiber.

12. The degradable composite material according to claim 11, wherein the thermostable chitinase is a thermostable chitinase originating in Pyrococcus furiosus.

13. A medical material manufactured from the degradable composite material according to claim 11.

14. A medical material manufactured from the degradable composite material according to claim 12.

15. The medical material according to claim 13, wherein the medical material is a wound dressing or medical prosthetic material.

16. The medical material according to claim 14, wherein the medical material is a wound dressing or medical prosthetic material.

17. A method for treating a wound, the method comprising the step of:
applying a sheet of degradable composite material sheet according to claim 11 to a wound site of a patient.

18. A method for suturing an incision site or a laceration site, the method comprising the step of:
suturing a degradable composite material fiber according to claim 11 into or on an incision site or a laceration site on a patient.
